(19) United States
(12) Patent Application Publication
(10) Pub. No.: US 2021/0379340 A1
(43) Pub. Date: Dec. 9, 2021

(54) BIOMEDICAL PRESSURE SENSOR

(71) Applicants: SOMNI CORPORATION B.V., Den Haag (NL); Millar, Inc., Houston, TX (US)

(72) Inventors: Anne STOKER, Pearland, TX (US); Germán Enrique KNOPPERS, Oude Leede (NL)

(73) Assignees: SOMNI CORPORATION B.V., Den Haag (NL); Millar, Inc., Houston, TX (US)

(21) Appl. No.: 17/288,064

(22) PCT Filed: Dec. 5, 2019
(86) PCT No.: PCT/EP2019/083827

§ 371 Date: Apr. 23, 2021

(30) Foreign Application Priority Data
Dec. 5, 2018 (NL) 2022134

(51) Int. Cl. A61M 25/06; G01L 11/02

(52) U.S. Cl. CPC A61M 25/0662; G01L 11/025; A61M 2025/0001; A61M 2205/3306; A61M 2025/0681

(57) ABSTRACT

A biomedical pressure sensor for measuring the pressure in a fluid includes an optical fiber having at least one measurement section arranged at a distance from a distal end of the optical fiber. The biomedical pressure sensor further includes a deforming member on the outer surface of the optical fiber at the location of the measurement section that is arranged for locally deforming the optical fiber under the influence of the applied pressure of the fluid to be measured. The measurement section is arranged for measuring said local deformation of the optical fiber.

The present invention relates to a biomedical pressure sensor comprising an optical fiber comprising at least one measurement section arranged at a distance from a distal end of the optical fiber, wherein the measurement section is arranged for locally measuring the deformation of the optical fiber. The invention further relates to a catheter provided with such a biomedical pressure sensors and a method for measuring pressure.

Existing (bio)medical pressure sensors are typically based on electro-mechanical principles. Often used biomedical pressure sensors are for instance air-charged catheters, which comprise tiny pressure-sensing air balloons to assess internal pressures. Alternatively, fluid-filled catheters, comprising a catheter with a balloon mounted on the tip, are also used. The balloon is filled with fluid, and the pressure is transmitted through the catheter and measured with an external-pressure transducer. Air-charged catheters act as an overdamped system, whereas fluid-filled catheters act as an underdamped system. Due to the dynamic behavior of these pressure sensors, accurate measurements of the dynamic pressure variations in the body, due to for instance the pumping action of the heart, can be hard to obtain.

On the other hand, the optical fiber pressure sensors have become increasingly common in the medical field. These pressure sensors are either based on the principle of interferometry, whereby a Fabry-Perot cavity is located on the tip of an optical fiber and enclosed by a miniature glass diaphragm to measure a deformation of the tip of the fiber. These pressure sensors are limited in measuring pressure at the tip of the fiber only.

It is a goal, amongst other goals, to provide an improved and/or efficient biomedical pressure sensor. This goal, amongst other goals, is met by a biomedical pressure sensor according to claim 1. More specifically, this goal, amongst other goals, is met by a biomedical pressure sensor for measuring the pressure in a fluid, wherein the biomedical pressure sensor comprises an optical fiber comprising at least one measurement section arranged at a distance from a distal end of the optical fiber, wherein the biomedical pressure sensor further comprises a deforming member on the outer surface of the optical fiber at the location of the measurement section that is arranged for locally deforming the optical fiber under the influence of the applied pressure of the fluid to be measured and wherein the measurement section is arranged for measuring said local deformation of the optical fiber. The deforming member of the biomedical pressure sensor will deform under the applied pressure, thereby introducing a strain in the optical fiber, which can be measured. The deforming member is thus arranged to act as a transducer for converting one physical quantity, such as a pressure that cannot directly be measured, to another physical quantity that can be directly measured, such as a local deformation of the optical fiber, at least at the location of the measurement section. Thereby, the pressure sensor is able to measure a pressure at a distance from the end of the fiber. Thus, a more versatile biomedical pressure sensor is obtained, whereby one is able to create a measurement section at any location along the optical fiber, such that a pressure can be measured not only at the end of the fiber, but also at any location along the length of the fiber.

It is noted that fluids in the framework of the invention comprise liquids and gasses and that a fluid is generally defined a substance that continually deforms (flows) under an applied shear stress. Preferably, the pressure sensor is however arranged to measure pressures in a liquid.

Different solutions can be employed for measuring the local deformation of the optical fiber at the location of the measurement section. For instance, a Fabry-Perot interferometer can be employed, which is based on the principle of interferometry, and typically has partially reflective glass optical flats that are spaced apart. If the relative distance between the partially reflective glass optical flats changes, due to for instance a local deformation of the optical fiber, the spectrum of the reflected light is also altered. Another option would be to apply a fiber Bragg grating (FBG) at the measurement section, wherein a type of distributed Bragg reflector is constructed in a short section (i.e. the measurement section) of an optical fiber that reflects particular wavelengths of light and transmits all others. If the measurement section is deformed, the properties of the grating also change, thereby leading to a shift in the wavelengths that are reflected. Note that these examples are not limiting.

In a preferred embodiment a deforming member extends at diametrical opposite locations on the optical fiber. Hereby an easier to produce optical pressure sensor is obtained, as the exact orientation of the deforming member with respect to the optical fiber and/or measurement section is of less importance. The deforming members may extend only at certain circumferential locations (seen along the circumference of the optical fiber) on said fiber, while other locations are not provided with a deforming member. A plurality of distinct deforming members may then be provided along the circumference of the fiber. Providing the deforming members at diametrical opposite locations then improves the measurement accuracy.

The deforming member is, in a preferred embodiment, a cylindrical member surrounding the optical fiber. By shaping the deforming member as a cylindrical member that surrounds the optical fiber at least at the location of the measurement section, the deforming member can easily and economically be made. Other shapes, such as a sphere, cuboid, hexagonal prism, triangular prism, etc. are also possible. A deforming member according to any of these shapes can, for instance, be formed through a casting or injection-molding process around the optical fiber, by providing a die with an appropriately shaped mold cavity. By including a suitably shaped though hole in the die, the optical fiber can be placed in the die before inserting the molten material of the deforming member whereby the hardened deforming member is directly located and attached to the optical fiber at the correct location. Another option would be to pre-form the deforming member in a single, or multiple pieces, and fixing the deforming member through gluing, or any other similar method for fixating the deforming member, to the optical fiber.

Preferably, the deforming member is arranged to deform the optical fiber in a substantially axial direction under the influence of the applied pressure. Due to the axial deformation of the deforming member, an axial uniform deformation of the optical fiber is imposed at the location of the measurement section, leading a shift in the wavelengths that are reflected by for example a Bragg reflector. In turn, the measurement results of the reflected light are easily interpreted, for instance in a suitably configured measurement unit which is arranged to be connected to the sensor.

In a preferred embodiment the outer surface of the deforming member is arranged to be in direct contact with the surrounding fluid. The applied pressure of the surrounding fluid works directly on the deforming member, which leads to an efficient transfer of the pressure of the fluid to the deforming member. Hence, a simple construction of the sensor is enabled, as there is no complex transfer path over different components of the sensor.

In a preferred embodiment, the deforming member is arranged to contract under the influence of an increase in applied pressure of the fluid. By arranging the deforming member such that it contracts (i.e. shortens) due the compression that is caused by the applied pressure, the measurement accuracy is improved. A correlation between the applied pressure and contraction of the deforming member can for instance be obtained, either theoretically through a model or through an initial calibration measurement. In case the deforming member is made from a linear-elastic material, or from a material that is only loaded in a region of its stress-strain relation wherein the material can be assumed to behave linear-elastic, a substantially linear relation between the applied pressure and the compression, or contraction, of the deforming element is obtained. This results in an easy to use sensor, whereby a simple linear interpolation between calibration points can be used for determining the pressure measured by the sensor. A suitable measurement unit can be configured to this end.

Preferably, the deforming member comprises end surfaces perpendicular to the axial direction, wherein the measuring section extends between the two end surfaces. As the measurement section extends in between the two end surfaces, it is fully enclosed by the deforming member. Thereby, the entire measurement section is substantially uniformly deformed, leading to reliable measurements. In a further preferred embodiment the biomedical pressure sensor is arranged such that the end surfaces are in contact with the fluid. In this case, the deforming member is over the full outer surface, or perimeter, in direct contact with the fluid and thereby the pressure acts over the full outer circumference, leading to an increased sensitivity of the sensor. Preferably, the end surfaces of the deforming member extend at or near the ends of the measuring sections. The length of the deforming member may for instance be equal to, or 5% or 10% larger than the length of the measurement section.

It is also preferred if the optical fiber comprises multiple measurement sections. Thereby, multiple pressure measurements can be done at various locations along the optical fiber using only a single optical fiber. The respective measurement sections are hereto preferably mutually separated at distances in the axial direction of the optical fiber. By creating a variable spacing between the different measurement sections, a versatile sensor-array can be obtained that is able to measure at different, predetermined, locations that are spaced over the length of the optical fiber. For example, FBGs provided in different measurement sections can have mutually different grating periods, such that each measurement section employs different frequencies in the light spectrum. Thereby the different measurements can be separated from each other using signal processing, for instance in the measurement unit connectible to the sensor.

Preferably, a deforming member is provided on the outer surface of the optical fiber at at least the locations of the measurement sections. As explained above, providing a deforming member improves the accuracy of said measurement section. Preferably, at least two or each measurement section of the pressure sensor according to an embodiment is provided with a respective deforming member. The respective deforming members are preferably mutually separated at distances in the axial direction of the optical fiber. As the deforming members are only required at the locations of the different measurement sections and can all be constructed similarly, a simple to produce and effective pressure sensor-array is obtained. Moreover, the end surfaces of the respective deforming members are preferably arranged to be in contact with the fluid. This again improves the sensitivity as explained above.

Alternatively, at least two or each of the respective measurement sections are enclosed in a continuous deforming member that is provided on the outer surface of the optical fiber. This option is beneficial in case the different measurement sections are closely spaced for obtaining distributed pressure measurements at a relatively high spatial resolution, as one only needs to arrange a single large deforming member on the optical fiber. Furthermore, it leads to a pressure sensor that is relatively simple in construction, as the deforming member can, for instance, be extruded around the optical fiber. It was experimentally verified that the cross-talk between the different measurement sections that are arranged with a single large deforming member is negligible. These two alternatives can also be combined on a single optical fiber, whereby closely spaced measurement sections, or groups of closely spaced measurement sections, are respectively arranged with a single, or multiple, larger deforming member(s), whereas the remainder of the measurement sections is arranged with individual deforming members.

In a preferred embodiment of the pressure sensor, the optical fiber further comprises a reference measurement section arranged for measuring a reference deformation of the optical fiber due to a temperature of a surrounding fluid, or due to the ambient temperature. As the pressure is converted to an induced strain on the optical fiber by the deforming element, the pressure read-out of the sensor is directly related to the elongation, or strain, measured in the measurement section. Due to the thermal expansion coefficient of the material of the deforming element and/or of the material of the optical fiber itself, together with the thermos-optic effect, i.e. the apparent optical elongation of the fiber due to the increase in temperature, part of the strain signal is also induced by a temperature difference with respect to the temperature at which calibration measurements (or simulations, or the like) have been performed. Hence, in order to correct the pressure measurements for the influence of the temperature of the surrounding fluid, a reference measurement section is added. The measurement unit may be arranged to correct for this pressure. Thereby, an accurate pressure reading can be obtained wherein potential errors caused by temperature differences have been minimized.

In a further preferred embodiment, the reference measurement section is arranged in close proximity to a measurement section and/or wherein the outer surface of the optical fiber at the location of the reference measurement section is in direct contact with the surrounding fluid. Hereby, the actual temperature can be measured near the measurement section. Especially in situations where temperature changes are experienced along the length of the optical fiber, it is important to perform the reference measurement in close proximity to the measurement section. Hereby, the error of the pressure measurement that is due to temperature can be minimized. A preferred embodiment of the biomedical sensor comprises a deforming member, wherein properties a, b, ν, F of the deforming member are such that S_{P2λ}≥0.10 fm/Pa, more preferably S_{P2λ}≥0.15 fm/Pa, wherein S_{P2λ} denotes the sensitivity of the biomedical pressure sensor, according to:

$${S}_{P 2 λ} ( a , b , v , E ) := [ \frac{{a}^{2} \cdot ( 2 v - 1 ) + {b}^{2} {v}_{quartz}}{E \cdot {a}^{2} - E \cdot {b}^{2} + {E}_{f} \cdot {b}^{2}} ] \cdot {S}_{ɛ2λ}$$

wherein a denotes the outer radius of the deforming member, b denotes the inner radius of the deforming member (and/or outer radius of the optical fiber), ν denotes the Poisson's ratio of the material of deforming member, ν_{quartz2} denotes the Poisson's ratio of material of the optical fiber, E denotes the Young's modulus of the material deforming member. E_{f} denotes the Young's modulus of the optical fiber, S_{ε2λ} a denotes the ratio of strain (ε) to the wavelength (λ). By dimensioning the deforming element such that the minimum sensitivity of the biomedical pressure sensor is obtained, a robust, practically usable and reliable sensor is obtained with an adequate signal-to-noise ratio, which can be combined with a highly sensitive measurement unit or interrogator for use in the medical field.

Preferably, an outer radius of the deforming member is no less than two times an inner radius of the deforming member, preferably no less than three times the inner radius of the deforming member, more preferably no less than five times the inner radius of the deforming member, most preferably no less than ten times inner radius of the deforming member. Thereby, the total force, due to the applied pressure, acting on the deforming member is sufficiently to deform the measurement section to allow for optically sensing the deformation of the measurement section. The inner radius of the deforming member preferably corresponds to the outer radius of the optical fiber.

It is preferred if the Young's modulus of the material of the deforming member is less than 10 GPa, preferably less than 5 GPa, more preferably less than 3 GPa, even more preferably less than 2 GPa, most preferably less than 1 GPa. A lower Young's modulus leads to more deformation in the deforming member, and subsequently the measurement section, and thereby to higher sensitivity of the pressure sensor.

In a preferred embodiment, the Poisson's ratio of the material of the deforming member is less than 0.5, more preferably less than 0.4, even more preferably less than 0.3, most preferably less than 0.2. In case the pressure is allowed to act on all the external surfaces of the deforming member, a lower Poisson's ratio leads to a higher sensitivity. The Poisson's ratio is a measure of the Poisson effect, which is the phenomenon in which a material tends to expand in directions perpendicular to the direction of compression. Hence, if a positive pressure is applied to the outer circumferential surface of the deforming element, the deforming element is urged to compress in the radial direction. As a result of the Poisson's effect, the deforming element tends to expand in the axial direction. At the same time, a positive pressure may also be acting on the end surfaces of the deforming element, urging the deforming element to compress in the axial direction and expand in the radial direction. A high Poisson's ratio leads a situation wherein these two mechanisms work against each other, thereby reducing the total amount of deformation of the deforming element and thus reducing the sensitivity of the pressure sensor. In case of a material with a low Poisson's ratio, the deforming member will compress in axial and radial directions without causing much interference and thus increasing the sensitivity of the pressure sensor.

The optical fiber has, in a preferred embodiment, an outer diameter of no more than 125 μm, preferably no more than 80 μm, more preferably no more than 50 μm, most preferably an outer diameter of around 25 μm or less. An optical fiber with a smaller outer diameter leads to an efficient pressure sensor, which can easily be applied in, for instance, narrow blood vessels or other locations with limited space. In addition, the small outer diameter leads to an unobstructed flow, such that placement of the sensor does not lead to pressure buildup.

In a preferred embodiment, the measurement section comprises a fiber Bragg grating (FBG). Fiber Bragg gratings can relatively easily be manufactured in optical fibers, while allowing for accurate measurements. In addition, multiple FBGs can be provided in a single optical fiber. By varying the grating period of the FBGs, each FBG works with a different frequency band in the light spectrum, thereby enabling that the measurements of the different FBGs can be separated by means of signal processing applied to the reflected light that is captured. A suitable measurement unit can be configured to this end.

In a preferred embodiment, the biomedical pressure sensor further comprises a housing that at least partially encloses (and/or covers) the optical fiber. Preferably the housing also at least partially encloses (and/or covers) the deforming member. A housing will shield the optical fiber from potential damages. In addition to this, the housing can provide for a smooth covering for the optical fiber and the deforming member that protrudes from the outer surface of the optical fiber, due to the smooth covering of the housing, the optical fiber can be moved around and positioned in narrow surroundings, while avoiding that protruding sections, such as the deforming member, get stuck. Preferably, the deforming member is arranged to move freely with the respect to the housing to allow the accurate measurements.

It is further preferred if the optical fiber is connected to the housing at a location at a distance from the measuring section. Any forces acting on the optical fiber due to this connection will thus be sufficiently far from where the pressure measurements are taken. The measurement section thereby experiences a minimum influence of the connection with the housing, such that accurate pressure measurements can be obtained. Alternatively, or additionally, the optical fiber is substantially stress-free mounted in the housing, in at least the axial direction, thereby avoiding that stresses are introduced in the optical fiber that can potentially affect the accuracy of the measurements. The optical fiber can, for instance, be mounted in the housing with a slight over-length with respect to the housing, which ensures that tensional stresses are avoided. As the optical fiber is preferably flexible, compression of the optical fiber is also minimized. This means that the fiber is mounted in the housing with slack.

As described above, it is preferred if the deforming member is in direct contact with the fluid of which the pressure is to be measured. Thereto, in a preferred embodiment, the housing comprises at least one opening extending between the interior and the exterior of the pressure sensor. The opening thereby allows for surrounding fluid to enter the interior of the pressure sensor, whereby the pressure of the surrounding fluid can be measured. It is further preferred if the opening is at a location of at least one measurement section, such that an outer surface of the deforming member is in direct contact with the fluid. This allows that the deforming member is deformed by the applied pressure of the fluid.

An embodiment of the invention further relates to a catheter comprising the biomedical sensor, wherein the catheter is an elongated member surrounding the optical fiber over at least the largest part of the optical fiber in the axial direction, wherein the catheter is arranged to be inserted in a body. A catheter allows for a safe and easy way of inserting the pressure sensor into the body. In addition, as the catheter surrounds the optical fiber over at least the largest part of the optical fiber, the pressure sensor can more easily be handled and the risk of damaging the optical fiber is substantially reduced.

In a preferred embodiment of the catheter, an inner surface of the catheter is in direct contact with the optical fiber and an outer surface of the catheter is arranged to be in direct contact with the fluid, wherein the deforming member is formed integrally with the catheter. The catheter is hereby formed integrally with the deforming member, whereby around the optical fiber a solid sleeve is formed over at least the largest part of the optical fiber, preferably even over the full length of the optical fiber. The deforming member preferably forms the outer surface of the catheter. The catheter allows for handling of the sensor (and/or the optical fiber), while also shielding it from damages. In addition, such a catheter with pressure sensor can, for instance, easily be formed through extruding a suitable material over the optical fiber. As the measurement sections are thereby also encapsulated by the substantially solid catheter, the deforming member that is associated with the measurement section is formed integrally with the catheter. This thus allows for a simple manufacturing process.

In a further preferred embodiment the elongated solid member is made from at least two materials, wherein at least the deforming member is made from a first material and wherein the remainder of the elongated solid member is made from a material different from the first material. In this embodiment the deforming member is still an integral part of the elongated solid member forming the catheter. Nonetheless, it allows for selecting a first material, which is highly suited for the deforming member, while selecting a different material, or different materials, for the remainder of the elongated solid member. In a production process one can first arrange the deforming members at the respective measurement locations on the fiber in a first material, after which the remainder of the fiber is encapsulated in a different material, or different materials, then the first material, or the other way around. Optionally, a material other than the first material can also be used to at least partially surround the deforming members. Thereby, a single material, which is for instance highly suitable for in vivo purposes, can be provided on the outer surface of the catheter. Also, if, for instance, a non-transparent, preferable colored, first material is used for the deforming members and a second, at least partially transparent, material is employed for the remainder of the elongated solid member, wherein the deforming members are covered by the second material, one can easily identify the locations of the measurement sections (i.e. the sensor-locations) along the catheter.

Alternatively, or additionally, the catheter according to a preferred embodiment comprises a housing and wherein the deforming member is arranged to be in open fluid communication with the fluid surrounding the catheter at the location of the measurement section in use. This allows that the surrounding fluid is in direct contact with the outer surface of the deforming member, such that the deforming member is deformed by the applied pressure of the fluid.

The catheter, according to a preferred embodiment, further comprises an elongated tubular sheath, wherein the biomedical pressure sensor is substantially enclosed by the elongated tubular sheath. By providing the catheter as an elongated tubular sheath, with a shape similar to the optical fiber and the deforming member in a preferred embodiment, the catheter closely fits the biomedical pressure sensor. Hence, a thin, smooth and flexible catheter is obtained that can easily, and with minimal trauma to any tissue, be inserted into the body. The catheter is preferably arranged such that the measurement section is able to measure a pressure in an environment surrounding the catheter at the location of the measurement section. Preferably, a peripheral wall of the elongated tubular sheath hereto comprises an opening, preferably at at least the location of the measurement section, thereby allowing for accurately measuring the pressure of the surrounding fluid.

The elongated tubular sheath of the catheter, in a preferred embodiment, comprises a plurality of tubular sections, whereby an end of a first tubular section is arranged at a distance from an end surface of the deforming member at a first end and wherein an end of a second tubular section is arranged at a distance from an end surface of the deforming member at a second end. A deforming member then extends between two tubular sections. It is then preferred if said first and second tubular sections are connected through a third tubular section for structural integrity. The end of the first tubular section and a first end of the third tubular section and the end of the second tubular section and a second end of the third tubular section may at least partially overlap. A peripheral wall of the third tubular section is preferably fluid permeable. This allows the measurement section to accurate measure the pressure via the respective deforming member. It is even more preferred if the peripheral wall of the third tubular section comprises at least one opening.

A catheter that is constructed in such a modular manner can be made more easily. Instead of a single elongated tubular sheath wherein holes need to be fabricated, or where by any suited other means, the measurement sections need to be enabled to measure the pressure of the surrounding fluid, one can provide multiple sections of tubular sheath for covering parts of the optical fiber in between measurement sections, while providing permeable sections tubular sheath for obtaining a direct pressure and/of fluid connection between the deforming member and the surrounding fluid.

The different sections of tubular sheath can, for instance, be connected by providing an overlap and the use of glue, suitable welding process, shrink-fits or any other suitable connection means. It is further noted that these third tubular sections can, for instance, be made from a permeable material, or non-permeable material in which case these sections are provide with holes and/or slits for enabling the pressure and/or fluid connection.

According to a preferred embodiment of the catheter, the deforming member is free to move with respect to the peripheral wall of the catheter and/or wherein, in use, fluid is free to move in between the outer surface of the deforming member and an inner surface of the peripheral wall of the catheter. By allowing the deforming member to freely move, accurate measurements can be obtained, as the influence due to potential contact between the deforming member and the catheter, such as for instance hysteresis caused by frictional effects, is minimized. Also, as fluid is free to move around all the outer surfaces of the deforming member, a uniform pressure acts on all the outer surfaces, preferably including the end surfaces, of the deforming member. Thereby, the measurement section can be substantially uniformly deformed, leading to reliable measurements. Also, as the deforming member is over the full outer surface, or perimeter, in direct contact with the fluid and thereby the pressure acts over the full outer circumference, this leads to an increased sensitivity of the sensor.

According to a preferred embodiment of the catheter, an outer diameter of the catheter is 8 Fr or less, preferably than 5 Fr or less, more preferably 3 Fr or less and most preferably 1 Fr or less. Such a very thin catheter can easily, and with minimal trauma, be inserted into the body, while at the same time having a minimal effect on the pressure measurements. In addition, measurements in the small veins and cavities of the body are enabled through the use of such a catheter with an optical pressure sensor. In addition, such a very thin catheter is suited for use within a guide catheter. A guide catheter can be surgically placed in a patient and allows for an easier insertion of multiple catheters, comprising a number of different instruments or sensor, and/or different surgical instruments in the human body. Hence, by placing the pressure sensor in a catheter that is arranged to be inserted in a guide catheter, along with other instruments and/or catheters, fewer incisions in the human body are required for placement of different sensors and/or surgical instruments.

The invention also relates to a method for measuring pressure, comprising of the steps of: providing a biomedical pressure sensor, comprising an optical fiber and at least one measurement section for locally measuring the deformation of the optical fiber, wherein the measurement section is positioned at a distance from a distal end of the optical fiber, a deforming member is arranged on the outer surface of the optical fiber at the location of the at least one measurement section and wherein the layer of material and optical fiber are connected and arranged to jointly deform in dependence of the applied pressure; projecting light in one end of the optical fiber; measuring the light reflected. The properties of the reflected light can then be used to determine a pressure at the measurement location. Preferably, the method also comprises the step of inserting the biomedical pressure sensor in a human or animal body. Hereby, pressures at different location is the body can easily, and with little trauma, be measured.

Note that the pressure sensor as has been described can be applied in other technical fields as well. The description, examples and embodiments of the pressure sensor that have been given can also be applied outside of the biomedical field. Such a pressure sensor can be applied in all cases where one needs to measure pressures in fluids. The proposed solution of a thin and robust sensor, that is able to measure pressure at multiple locations along a single fiber, is thus useful in many fields of technology. Instead of providing a catheter for use in the biomedical field, a more simple housing can be applied in these cases.

The present invention is further illustrated by the following Figures, which show a preferred embodiment of the device and method according to the invention, and are not intended to limit the scope of the invention in any way, wherein:

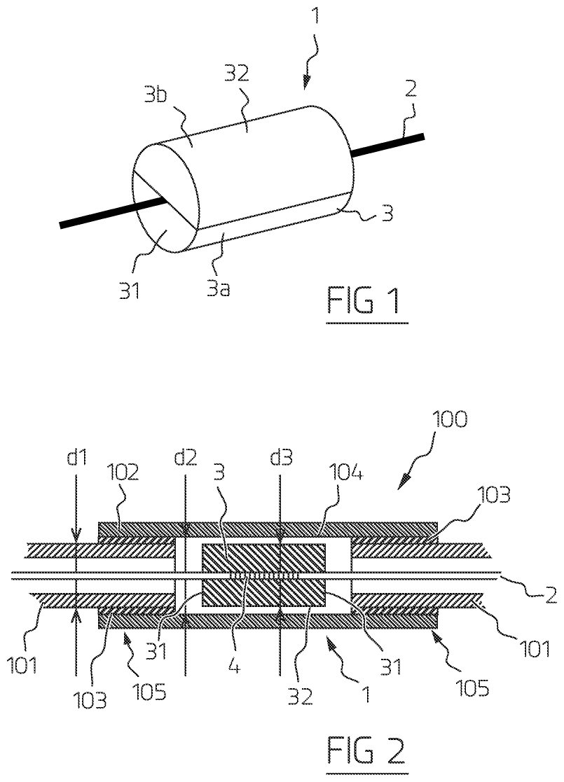

FIG. 1 is a perspective view of an embodiment of the pressure sensor according to the invention.

FIG. 2 is a cross-sectional view of an embodiment of the pressure sensor according to the invention.

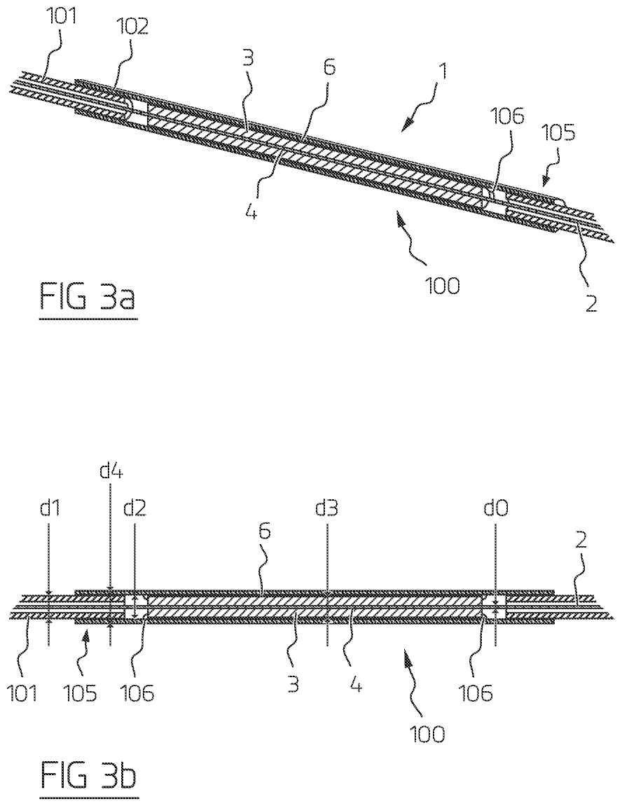

FIGS. 3a and 3b show in a perspective and a cross-sectional view a preferred embodiment of the sensor according to the invention.

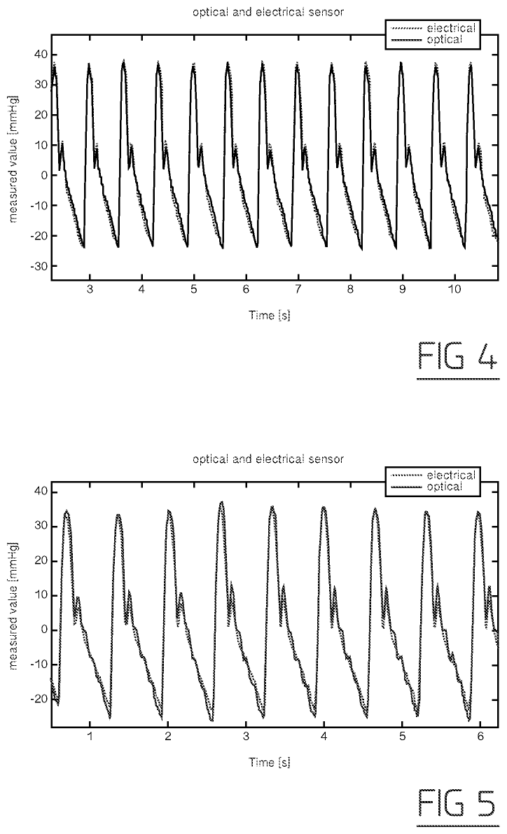

FIGS. 4 and 5 show the results of one-to-one comparisons between a pressure sensor according to the invention and an electrical pressure sensor.

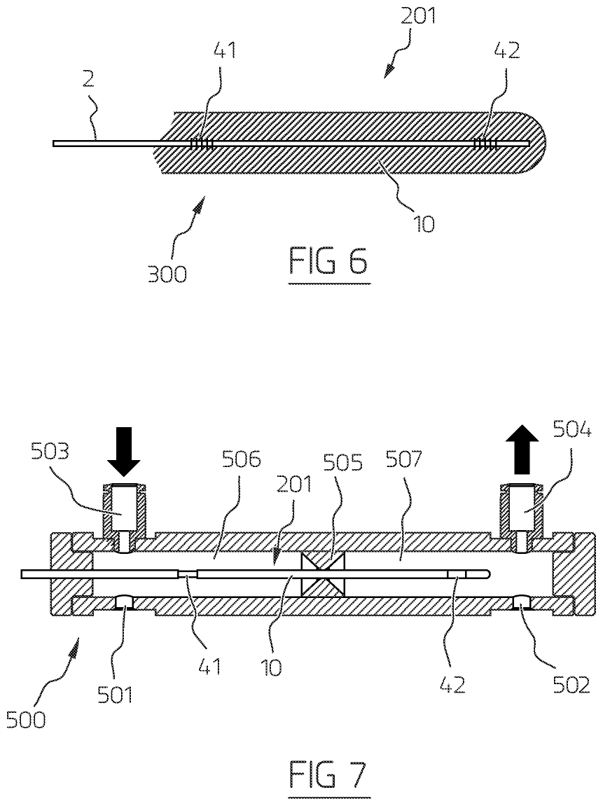

FIG. 6 shows the cross-section of an embodiment of a pressure sensor within a solid catheter according to the invention.

FIG. 7 schematically shows the test-setup for evaluating the cross-talk between the different measurement sections of the pressure sensor shown in FIG. 6.

In FIG. 1 a pressure sensor 1 is schematically shown. An optical fiber 2 is enclosed by a cylindrical member 3, that is formed by gluing (or fixing by means of any suited method) two parts 3a,3b around the optical fiber 2. The cylindrical member 3 shown in FIG. 1 acts as the deforming member and essentially covers the measurement section, comprising a grating (not visible in FIG. 1), that is present on the optical fiber 2. The pressure sensor 1 as shown is suitable for direct use. By placing it in a fluid of which the pressure needs to be determined, the fluid comes into direct contact with the full outer surface of the part of the sensor that is submerged. The fluid, and thereby also its associated pressure, is direct distributed on the full outer circumferential surface 32 and on both end surfaces 31 of the deforming member 3. The pressure leads to, depending on the material used for the deforming member, to a contraction or expansion in the longitudinal, or axial, direction 1 of the deforming member 3. As the optical fiber 2 is fixed to the deforming member 3 over substantially the full length of the deforming member 3, the contraction and or expansion leads to a substantially equal deformation in the measurement section of the optical fiber 2. This change of length is detected through the changing wavelengths that are reflected by the Fiber Bragg Grating. A suitable measurement unit can be configured to this end.

FIG. 2 again shows that the optical fiber 2 is enclosed by a cylindrical member 3. In this cross-sectional view, the grating 4 of the fiber Bragg grating making up the measurement section can clearly be identified. The cylindrical member 3 fully encloses the grating 4, thereby ensuring that the measurement section, and thus the grating 4, is uniformly deformed by the pressure applied. The optical fiber 2 and cylindrical member 3 are enclosed by a catheter 100 that forms the housing of the pressure sensor 1. The catheter 100 is constructed from multiple sections. The catheter is mostly made up of the main sections 101 that enclose parts of the optical fiber 2 that are spaced apart from the measurement sections, which are encapsulated by the cylindrical members 3. Around the location of the cylindrical member 3, the sensor 1 is enclosed by a fluid permeable section 102. Such permeable section 102 is either made from a permeable material, or is provided with holes for enabling direct contact between the fluid of which the pressure is to be determined and the cylindrical member 3. Overlapping regions 105 between the main sections 101 and permeable section 102 allow for connecting these by means of a glue layer 103, or any suitable method for connecting the sections. The catheter 100 is thus made from a series of interconnected sections of tubular sheath. Note that, apart from the here shown catheter, different types of housings can be placed around the pressure sensor 1, or around the deforming member 3. A main purpose of such a housing is for instance to shield the more fragile part of the sensor, or to provide for a better and safer way of handling the pressure sensor 1.

It is furthermore noted that the outer diameter d₁ of the main section 101 is substantially equal, or only slightly larger, than the outer diameter d₃ of the cylindrical member 3. The inner diameter d₂ of the permeable section 102 is somewhat larger than the outer diameters d₁ and d₃. Due to this construction, the cylindrical member 3 is free to move with respect to the inner wall 104 of the permeable section 102. Also, as the main sections 101 are spaced at a distance from the end surfaces 31 of the cylindrical member 3, the cylindrical member is, in general, free to move with respect to the catheter 100. In addition, the sections 101, 102 making up the catheter 100 can easily be assembled and connected. Due to the fact that diameters d₁ and d₃ only slightly differ, the cylindrical member 3 can only be fixed to the optical fiber 2 during assembly, as main sections 101 cannot slide over the cylindrical members 3. Hence, when assembling the catheter (from left to right) one first needs to slide optical fiber into (left) main section 101, fix the cylindrical member 3 to the optical fiber 2 at the location of the grating 4 and after this mount the (right) main section 101 and permeable section 102 before fixing all the sections 101, 102 together for obtaining the assembled catheter comprising the pressure sensor 1.

As the optical fiber 2 is connected (not shown) at a distance from the grating 4 (making up the measurement section), the influence, due to connecting forces introduced into the fiber 2, on the pressure measurements is minimized.

Note that even though the deforming member is, in the current example, a cylindrical member 3, different types of shapes, such as a sphere, cuboid, hexagonal prism, triangular prism, etc. are also possible. The catheter can similarly be formed from tubular sections with different, corresponding, cross-sections.

FIGS. 3a and 3b show cross-sections of a catheter 100 comprising the pressure sensor 1. The permeable section 102 comprises multiple openings 106, wherein an opening 106 can either be formed by, for instance, circular holes or slits extending in the axial direction of the permeable section 102 over substantially the full length of the cylindrical member 3.

The sensitivity of the pressure sensor 1 is for a large part determined by the outer diameter d₀ of the optical fiber 2, and more specifically, by the ratio between outer diameter d₀ of the fiber 2 (which corresponds to the inner diameter of the cylindrical member 3) and the outer diameter d₃ of the cylindrical member 3, such that d₃/dₙ should be maximized for optimizing the sensitivity. The outer diameter d₃ is in turn limited by the maximum allowable outer diameter d₄ of the permeable section 102. Hence, the optimal design requires an as small as possible gap 6 between the outer diameter d₃ and the inner diameter d₂, such that d₃/d₀ can be maximized, while still ensuring that the cylindrical member 3 is free to move within the catheter 100. In addition, the fluid of which the pressure needs to be measured has to be able to flow around the full circumference of the cylindrical member 3, such that the pressure is uniformly transferred to the cylindrical member 3.

In FIG. 4 and FIG. 5 results of a one-to-one comparison between a traditional electro-mechanical pressure sensor and the optical pressure sensor 1 according to the invention are shown. The results were recorded at approximately 10 kHz and band pass filtered between approximately 1 and 100 Hz. In the test-setup the pulsating rhythm of a heart-beat has been simulated, whereby simultaneous pressure measurements have been performed using these to pressure sensors. The pressure sensor 1, according to FIG. 1, was fitted with a FBG in the 50 μm optical fiber 2. The cylindrical deforming member 3 had a 1 mm outer diameter and was manufactured by gluing two ABS parts around the optical fiber 2. FIG. 4 shows the results for the sensor 1 without a housing. For the results shown in FIG. 5, the optical sensor was fitted with a housing as shown in FIG. 2. Both figures show an excellent correspondence with the electro-mechanical reference measurements.

FIG. 6 shows, in a cross-sectional view, an embodiment of the pressure sensor 201 according to the invention. The basic ingredients of the sensor are the same as in the earlier embodiment. In this embodiment the optical fiber 2 comprising two grating sections 41, 42; and a deforming member, which is a continuous cylindrical member 10 encapsulating both grating sections 41, 42 and, at least, the part of the optical fiber 2 that is in between the first grating section 41 and the second grating section 42. The continuous cylindrical member 10 thus forms the deforming members for both grating sections 41, 42. The pressure sensor 201 can be placed in a tubular sheath, forming the housing, as has been shown in FIGS. 1-3. However, the continuous cylindrical member 10 can also be extended over substantially the full length of the optical fiber 2, thereby also serving as the housing (or catheter) 300. This obviously leads to a less complex production process, as the catheter 300 can in this case be disposed on the optical fiber 2 through, for instance, an extrusion process. Thereby, the cylindrical member 10 essentially acts as a deforming member, housing and catheter at the same time. The cylindrical member 10 is, when in use, thus in direct contact with the fluid of which the pressure is to be determined. In that case it is vital that, for at least the biomedical application, the material of which the cylindrical member 10 is made, is suited to for safe use in the body. Such a material is for instance non-toxic and can be easily sterilized.

FIG. 7 shows the test-setup 500 that has been used to verify that the sensor 201 shown in FIG. 6 experiences only limited, to virtually no, cross-talk between the first 41 and second 42 grating section. A fluid flows from inlet 503 to outlet 504, thereby passing through choke 505 for generating the pressure difference between low pressure chamber 507 and high pressure chamber 506. Cavities 501 and 502 have been fitted with pressure sensors for taking reference measurements. Optical pressure sensor 201 is fitted in the setup such that the second grating section 42 is located in the low pressure chamber 507 and first grating section 41 is located in the high pressure chamber 506. The optical fiber 2 and the grating sections 41, 42 have all been enclosed in the continuous cylindrical member 10, which forms the solid catheter. The results (not shown) have shown that accurate pressure measurements, with negligible cross-talk between the different measurement sections, can be obtained using the optical pressure sensor 201.

The sensor shown in FIG. 7 can also be produced using different materials. Instead of encapsulating the outer surface of the optical fiber 2 in a single material, as was shown for instance in FIG. 6, one can decide to make the deforming members 41, 42 from a second, different material than the remainder of the cylindrical member 10. In such a way, one can optimize for the material properties of the deforming members 41, 42, while being less constrained due to other, for instance, practical reasons, such as costs or manufacturability over long lengths. Alternatively, one can first manufacture the deforming member 41, 42 on the optical fiber 2, after which the remainder of the optical fiber is packaged in a second, different material. Hereby it is even possible to package the deforming member 41, 42 in the second, different material. In case the material for the deforming members 41, 42 is not suitable for direct use in medical applications, these sections can be shielded using the second, different material from which the remainder of the cylindrical member 10 is made. This packaging, or covering, operation can easily be performed by, for instance, an extrusion process. If the second, different material is, for instance, a transparent material, one can easily verify the location of the deforming members 41, 42, and thus the measurement sections, over the full length of the pressure sensor 201. Even the deforming members 41, 42 can be made from different materials if desired.

Note that this use of different materials for different purposes can be applied to all of the embodiments shown. Also, the present invention is not limited to the embodiment shown, but extends also to other embodiments falling within the scope of the appended claims.

1-39. (canceled)

40. A biomedical pressure sensor for measuring the pressure in a fluid, comprising:
an optical fiber having at least one measurement section arranged at a distance from a distal end of the optical fiber, and
a deforming member on the outer surface of the optical fiber at the location of the measurement section that is arranged for locally deforming the optical fiber under the influence of the applied pressure of the fluid to be measured,
wherein the measurement section is arranged for measuring said local deformation of the optical fiber.

41. The biomedical pressure sensor of claim 40, wherein the optical fiber is fixed to the deforming member over substantially the full length of the deforming member

42. The biomedical pressure sensor of claim 40, wherein a deforming member extends at diametrical opposite locations on the optical fiber.

43. The biomedical pressure sensor of claim 40, wherein the deforming member is a cylindrical member surrounding the optical fiber.

44. The biomedical pressure sensor of claim 40, wherein the outer surface of the deforming member is arranged to be in direct contact with the surrounding fluid.

45. The biomedical pressure sensor of claim 40, wherein the deforming member is arranged to deform the optical fiber in a substantially axial direction under the influence of the applied pressure and/or wherein the deforming member is arranged to contract under the influence of an increase in applied pressure of the fluid.

46. The biomedical pressure sensor of claim 40, wherein the deforming member comprises end surfaces perpendicular to the axial direction, wherein the measuring section extends between the two end surfaces, and wherein the biomedical pressure sensor is arranged such that end surfaces are in contact with the fluid.

47. The biomedical pressure sensor of claim 40, wherein:
the optical fiber comprises a plurality of measurement sections,
a deforming member is provided on the outer surface of the optical fiber at at least the locations of the measurement sections,
the respective measurement sections are mutually separated at distances in the axial direction of the optical fiber,
wherein each measurement section is provided with a deforming member, and
wherein the respective deforming members are mutually separated at distances in the axial direction of the optical fiber, or at least two of the respective measurement sections are enclosed in a continuous deforming member that is provided on the outer surface of the optical fiber.

48. The biomedical pressure sensor of claim 40, wherein the optical fiber further comprises a reference measurement section arranged for measuring a reference deformation of the optical fiber due to a temperature of a surrounding fluid, and
wherein the reference measurement section is preferably arranged in close proximity to a measurement section and/or wherein the outer surface of the optical fiber at the of the reference measurement section is in direct contact with the surrounding fluid.

49. The biomedical pressure sensor of claim 40, wherein properties a, b, ν, E of the deforming member are such that S_{P2λ}≥0.10 fm/Pa, wherein S_{P2λ} denotes the sensitivity of the biomedical pressure sensor, according to the formula:
$${S}_{P 2 λ} ( a , b , v , E ) := [ \frac{{a}^{2} \cdot ( 2 v - 1 ) + {b}^{2} {v}_{quartz}}{E \cdot {a}^{2} - E \cdot {b}^{2} + {E}_{f} \cdot {b}^{2}} ] \cdot {S}_{ɛ2λ}$$
wherein a [m] denotes the outer radius of the deforming member, b [m] denotes the inner radius of the deforming member (and/or outer radius of the optical fiber), ν [−] denotes the Poisson's ratio of the material of deforming member, ν_{quartz} [−] denotes the Poisson's ratio of material of the optical fiber, E [Pa] denotes the Young's modulus of the material deforming member, E_{f} [Pa] denotes the Young's modulus of the optical fiber, and S_{ε2λ} denotes the ratio of strain (ε) to the wavelength (λ).

50. The biomedical pressure sensor of claim 40, wherein:
an outer radius of the deforming member is no less than two times an inner radius of the deforming member;
the Young's modulus of the material of the deforming member is less than 10 GPa;
the Poisson's ratio of the material of the deforming member is less than 0.5 and/or;
the optical fiber has an outer diameter of no more than 125 μm.

51. A catheter comprising:
a biomedical sensor for measuring the pressure in a fluid, wherein the biomedical pressure sensor comprises an optical fiber having at least one measurement section arranged at a distance from a distal end of the optical fiber, wherein the biomedical pressure sensor further comprises a deforming member on the outer surface of the optical fiber at the location of the measurement section that is arranged for locally deforming the optical fiber under the influence of the applied pressure of the fluid to be measured and wherein the measurement section is arranged for measuring said local deformation of the optical fiber,
wherein the catheter is an elongated member surrounding the optical fiber over at least the largest part of the optical fiber in the axial direction, and
wherein the catheter is arranged to be inserted in a body.

52. The catheter of claim 51, wherein an inner surface of the catheter is in direct contact with the optical fiber and an outer surface of the catheter is arranged to be in direct contact with the fluid to be measured, and wherein the deforming member is formed integrally with the catheter.

53. The catheter of claim 51, wherein the elongated solid member is made from at least two materials, and wherein at least the deforming member is made from a first material and wherein the remainder of the elongated solid member is made from a material different from the first material.

54. The catheter of claim 51, wherein the catheter further comprises an elongated tubular sheath, wherein the biomedical pressure sensor is substantially enclosed by the elongated tubular sheath and the catheter is arranged such that the measurement section is able to measure a pressure in an environment surrounding the catheter at the location of the measurement section, and wherein a peripheral wall of the elongated tubular sheath comprises an opening at at least the location of the measurement section.

55. The catheter of claim 54, wherein the elongated tubular sheath comprises a plurality of tubular sections, wherein an end of a first tubular section is arranged at a distance from an end surface of the deforming member at a first end and wherein an end of a second tubular section is arranged at a distance from an end surface of the deforming member at a second end.

56. The catheter of claim 55, wherein said first and second tubular sections are connected through a third tubular section such that the end of the first tubular section and a first end of the third tubular section and the end of the second tubular section and a second end of the third tubular section at least partially overlap, and wherein a peripheral wall of the third tubular section is fluid permeable.

57. The catheter of claim 51, wherein an outer diameter of the catheter is 8 Fr or less.

58. A method for measuring pressure of a fluid, comprising:
providing a biomedical pressure sensor for measuring the pressure in the fluid, wherein the biomedical pressure sensor comprises an optical fiber comprising at least one measurement section arranged at a distance from a distal end of the optical fiber, wherein the biomedical pressure sensor further comprises a deforming member on the outer surface of the optical fiber at the location of the measurement section arranged for locally deforming the optical fiber under the influence of the applied pressure of the fluid to be measured and wherein the measurement section is arranged for measuring said local deformation of the optical fiber;
projecting light in one end of the optical fiber; and
measuring the light reflected.

59. The method of claim 58, further comprising inserting the biomedical pressure sensor in a human or animal body.
